(19) United States
(12) Patent Application Publication
(10) Pub. No.: US 2024/0350450 A1
(43) Pub. Date: Oct. 24, 2024

(54) COMPOSITION COMPRISING a-KETOGLUTARIC ACID (a-KG) AND 5-HYDROXYMETHYL-2-FURFURAL (5-HMF) TO IMPROVE HEMOGLOBIN-MEDIATED OXYGEN SUPPLY

(71) Applicant: CYL GmbH, Pöllau (AT)

(72) Inventors: Reinhold Wintersteiger, Graz-Mariatrost (AT); Chrisitian Bücherl-Harrer, Raaba Lamberg (AT)

(21) Appl. No.: 18/687,601

(22) PCT Filed: Aug. 30, 2022
(86) PCT No.: PCT/EP22/74104

§ 371 Date: Feb. 28, 2024

(30) Foreign Application Priority Data
Aug. 30, 2021 (EP) 21193855.0

(51) Int. Cl. A61K 31/341; A61K 31/194; A61P 7/00

(52) U.S. Cl. CPC A61K 31/341; A61K 31/194; A61P 7/00

(57) ABSTRACT

The present invention relates to a method of treating or preventing a disease or condition that benefits from an increased hemoglobin-mediated oxygen supply comprising administering to a subject a pharmaceutical composition comprising α-ketoglutaric acid (α-KG) and 5-hydroxymethyl-2-furfural (5-HFM).

The present invention relates to a pharmaceutical composition comprising α-ketoglutaric acid (α-KG) and 5-hydroxymethyl-2-furfural (5-HMF), for use in the treatment and/or prevention of a disease or condition that benefits from enhanced hemoglobin-mediated oxygen delivery. The description cites a plurality of documents, including patent applications, patents and instructions for use from various manufacturers. The disclosure content of each of these documents, although not considered relevant to the patentability of the present invention, is hereby incorporated into the disclosure content of the application. In particular, all documents referred to in these documents are also included in the disclosure content of the application, to the same extent as if each of these documents had been specifically incorporated by reference into the disclosure content of the present application.

Red blood cells (erythrocytes) are those blood cells whose main task is to transport the inhaled vital oxygen in the lungs through the blood vessels to the organs and tissues of the body. The red blood cells fulfill their task by the red blood pigment they contain, hemoglobin (Hb).

Hemoglobin is a tetrameric protein that can reversibly bind four oxygen molecules via an allosteric mechanism. In the blood, hemoglobin is present in a balance between two allosteric structural states. In the “T” state (tense), hemoglobin is deoxygenated. In the “R” state (relaxed), hemoglobin is oxygenated.

The oxygen dissociation curve (ODC, or “oxyhemoglobin dissociation curve”), or referred to as oxygen binding curve of hemoglobin (Hb), is a graphical representation of the relationship between the oxygen partial pressure (PO₂) and the oxygen saturation (SO₂) of Hb, wherein SO₂ corresponds to the percentage of oxygen-saturated Hb (oxyhemoglobin) in the total hemoglobin. The typically sigmoid curve shape of the ODC reflects the cooperativity of the reversible binding of four oxygen molecules to the tetrameric hemoglobin molecule. This means that the binding of one molecule of oxygen induces a cooperative conformational change of the Hb, transforming the so-called “tense state” into the “relaxed state”, making it easier for the hemoglobin molecule to bind further oxygen. The ODC assigns a specific oxygen saturation of hemoglobin to each oxygen partial pressure (PO₂): a low PO₂ leads to a decrease in saturation. In the middle region of the curve, the increase in PO₂ leads to an almost linear increase in oxygen saturation. At a certain PO₂ the Hb is saturated. A further increase in the oxygen partial pressure does not lead to any further significant increase in oxygen saturation.

The most important parameters to describe the ODC are the P50 value (the oxygen partial pressure (PO₂) at which 50% saturation of the Hb with oxygen (O₂) occurs; a measure of the O₂ affinity of Hb) and the Hill coefficient that represents the maximum slope of the ODC, and thus a measure of the cooperativity of oxygen binding to Hb (2).

The course of the ODC curve is influenced by numerous factors (3), which lead either to an increase in the HbO₂ affinity and thereof associated leftward shift of the ODC, or to a reduction of the HbO₂ affinity and thereof associated rightward shift of the ODC (4), wherein in general, a rightward shift favors oxygen discharge and a leftward shift favors oxygen loading.

Known physiological factors that lead to a rightward shift of the ODC are for example increased carbon dioxide concentrations, low (acidic) pH, increased 2,3-diphosphoglycerate (2,3-DPG, also known as 2,3-biphosphoglyceric acid (2,3-BPG)) concentration, and/or increased body temperature. This ultimately contributes to an improved oxygen delivery to metabolically active tissue, where oxygen and glucose are metabolized to carbon dioxide and organic acids.

A variety of diseases and conditions are known that are associated with an inadequate supply of oxygen, wherein these diseases or conditions are usually either due to an impaired oxygen uptake in the lungs or an impaired supply of oxygen-providing blood. Apart from (patho-) physiological causes, external factors, e.g. low air pressure at high altitudes or low oxygen content in the air, can lead to insufficient oxygen supply and therewith associated health complications. In addition, it is known that an efficient oxygen supply is fundamental for maintaining all physiological functions and thus for the physical and mental performance.

It was therefore proposed that influencing the allosteric balance of hemoglobin, and thus its oxygen-binding and -releasing properties, is a viable means for the treatment and/or prevention of a wide variety of such diseases or conditions, as well as a means of enhancing the physical and/or mental performance during physical exercise and at work.

It was suggested that influencing hemoglobin to a state of lower oxygen affinity, and the therewith associated rightward shift of the ODC, could be particularly beneficial for those diseases and conditions in which the tissues suffer from insufficient oxygen supply. It is assumed that the oxygen loading of Hb in the lungs would be impeded by the reduced oxygen affinity, but that the oxygen discharge in the peripheral tissue would be promoted overall.

In contrast, influencing hemoglobin by stabilizing the R state to a state of enhanced oxygen affinity, and the therewith associated leftward shift of the ODC, is considered particularly advantageous for the treatment of sickle cell anemia. It was shown that stabilizing the R state of the sickle cell hemoglobin (Hb-S) underlying this hereditary disease can counteract erythrocyte clumping associated with the T state, which is prone to polymerization, and the associated vascular obstruction (sickling).

On the other hand, it has been discussed in the past that substances that cause an enhanced oxygen affinity and the therewith associated leftward shift of the ODC lead to an improved oxygen loading, but to a more difficult oxygen discharge and therefore would not contribute to an improvement in the oxygen supply in the peripheral tissue. However, studies elucidated that the efficiency of oxygen discharge is primarily determined by the ODC curve: It was shown that substances which, on the one hand, increase the oxygen affinity of Hbs (i.e. cause a leftward shift of the ODC) but, on the other hand, preserve the sigmoid ODC curve shape, contribute to an overall improved oxygen supply.

For example, the micronutrient 5-hydroxymethyl-2-furfural (5-HMF) was identified as an allosteric modulator of Hb, increasing its affinity for oxygen. It was detected in an animal model of severe hypoxia that the HbO₂ affinity enhanced by 5-HMF can protect against hemodynamic instability and maintain microvascular oxygenation (8). In another study, it was shown that in pigs exposed to hypoxia, a 5-HMF-induced enhancement in HbO₂ affinity resulted in improved arterial oxygen saturation (SO₂) and attenuation of the hypoxia-associated increase in pulmonary arterial pressure (9). Similarly, 5-HMF-induced increases in HbO₂ affinity were detected in human subjects exposed to hypoxia as well as in sickle cell anemia patients (10).

Not least since the emergence and global spread of COVID-19 and the therewith associated respiratory complications, there is a constant need for new, alternative and improved therapeutic agents for the treatment and/or prevention of diseases and conditions that would benefit from improved hemoglobin-mediated oxygen delivery. In addition, there is also a growing demand for dietary supplements to optimize and enhance mental and physical performance during physical exercise and at work.

These problems were solved by the present invention according to the claims and the following specification and examples.

In a first aspect, the present invention relates to a pharmaceutical composition comprising α-ketoglutaric acid (α-KG) and 5-hydroxymethyl-2-furfural (5-HMF) for use in the treatment and/or prevention of a disease or condition that benefits from an enhanced hemoglobin-mediated oxygen delivery.

5-hydroxymethyl-2-furfural (5-HMF), also known as hydroxymethylfurfural (HMF), 5-(hydroxymethyl) furfural or 5-oxymethylfurfurol, is an aldehyde and furan compound which, e.g., forms in the thermal decomposition of sugar or carbohydrates and can be detected in many foods such as, e.g., in fruit juices or honey.

α-ketoglutaric acid (α-KG), also known as 2-oxoglutaric acid or 2-oxopentanedioic acid, is a dicarboxylic acid derived from n-pentane that carries an additional carbonyl group on the α-C atom. α-KG forms colorless, almost odorless crystals. α-KG is a naturally occurring, nitrogen-free portion of the amino acids glutamine and glutamic acid. α-KG, which is present in the aqueous environment within a cell as an anion (α-ketoglutarate), is an intermediate in the energy metabolism during ATP production in cells via the citrate cycle. α-KG is a stronger radical scavenger (RONS) than vitamin C in the equivalent dose and acts as a nitrogen regulator in metabolism. For purposes of the present disclosure, the terms α-ketoglutaric acid (α-KG) and α-ketoglutarate are used interchangeably.

In the context of the present invention, it was surprisingly found that the combined use of 5-HMF and α-KG leads to an enhancement in the oxygen affinity of hemoglobin while maintaining a sigmoid ODC curve shape. As shown in the Examples, the effects measured for the combination of 5-HMF and α-KG exceed those of the individual substances, from which a synergistic effect of the combination of 5-HMF and α-KG with regard to the allosteric modulation of hemoglobin can be concluded. This synergism is underlying the present invention.

The combination of 5-HMF and α-KG was already known as such and commercially available as a dietary supplement (Sanopal®). Thus, in a previous study by the inventors, it was reported that the administration of a combination of 5-HMF and α-KG in healthy subjects who exercised by cycling at a simulated altitude of 3500 m (FiO₂=13.5%), led to an increase in peripheral oxygen saturation (SpO₂) (12). In another study, it was reported that the combined administration of 5-HMF and α-KG as pre-operative oral micronutrient supplementation to lung cancer patients resulted in an enhanced maximum oxygen uptake (VO₂max) and a shortened hospitalization time after surgery (lobectomy with one-lung ventilation). In both studies, the likely cause underlying the observed effect was the known property of the combination 5-HMF and α-KG as a means of reducing oxidative stress, particularly tissue damage caused by it. However, an effect of said combination on the oxygen-binding properties of hemoglobin, as first disclosed in connection with the present invention, was completely unknown.

As used herein, the term “enhanced hemoglobin-mediated oxygen delivery” means that the combination of 5-HMF and α-KG results in a leftward shift of the hemoglobin oxygen saturation curve (ODC), wherein compared to a reference (e.g., a reference blood sample), said leftward shift can be measured in the absence of the combination. Various methods for measuring an ODC are routinely known to those skilled in the art. For example, the ODCs disclosed in the experiments of the present specification were determined with the in vitro method described by Woyke et al. (Ref: 20).

Moreover, it is to be preferably understood that the leftward shift of the ODC occurs while a sigmoid curve shape is maintained.

The term “disease that benefits from an enhanced hemoglobin-mediated oxygen delivery” as used herein includes all such diseases, disease symptoms and/or disorders that are characterized and/or caused by inadequate oxygen supply and/or for the alleviation or healing of which an improved hemoglobin-mediated oxygen delivery is beneficial.

The term “condition that benefits from an enhanced hemoglobin-mediated oxygen delivery” as used herein includes all those conditions or disorders prior to a detectable occurrence of a disease symptom or pathological change that is characterized and/or caused by a lack of oxygen supply and/or could lead to illnesses or pathological changes if left untreated.

In any event, the term that a disease or condition “benefits” from treatment and/or prevention shall be interpreted in such a way that the disease or condition is alleviated by said treatment or the risk of developing such disease or condition is reduced through prevention or even eliminated.

Although the therapeutic/preventive uses disclosed herein primarily target their application on humans (i.e., human individuals/patients), the present disclosure also includes applications in the veterinary field, i.e., on animals, in particular horses, dogs, farm animals (e.g., cattle, pigs, goats, poultry) and pack animals.

It is understood that the pharmaceutical composition according to the invention may have a pharmaceutically acceptable formulation. Pharmaceutically acceptable formulations are well known in the art. For example, see the paper by Rowe et al. Handbook of Pharmaceutical Excipients (6^{th} edition), R. C. Rowe, P. J. Sheskey, M. E. Quinn. Pharmaceutical Press, London, 2009.

The pharmaceutical composition according to the invention may further contain additives. These comprise any compound or composition that is advantageous for use according to the invention, including water, salts, binders, solvents, dispersants, buffers (particularly physiological buffers such as, e.g., Ringer's solution or phosphate-buffered saline (PBS)), stabilizers and other substances commonly used in connection with the formulation of drugs. The pharmaceutical composition may also include preservatives and other additives such as, e.g., antimicrobial compounds, antioxidants, complexing agents and inert gases.

Particularly preferred additives are water, sweeteners (e.g., sugar, preferably sucrose, glucose or dextrose, or sugar substitutes), magnesium chloride, and/or acidity regulators such as, e.g., potassium hydroxide and/or sodium hydroxide. Trace elements, vitamins, amino acids and/or plant extracts can also be used as additives. Further particularly preferred additives are all those agents that are required or beneficial for blood formation, such as, e.g., the vitamins B₁₂ (cobalamin), B₉ (folic acid) and/or B₆, the trace elements iron and/or selenium, and the amino acid methionine.

According to a preferred embodiment, the composition according to the invention contains α-KG and 5-HMF as the sole combination of active ingredients (i.e., without other pharmaceutically active ingredients). However, according to an alternative embodiment, the pharmaceutical composition can contain additional active ingredients, in particular those that can be beneficial and advantageous for the respective intended use. Examples include blood thinners, anticoagulants (e.g., heparin), blood pressure lowering agents, antibiotics, virostatics, anti-inflammatory substances (e.g., glucocorticoids), chemotherapeutics, immunosuppressants (e.g., cyclosporins or tacrolimus), proteins and/or peptides (including peptide hormones such as, e.g., angiotensin).

The administration of the pharmaceutical composition according to the invention is not limited to a specific method (mode of administration/dosage form) and can be oral or parenteral, e.g., intravenous, intraperitoneal, subcutaneous, intramuscular, local, intranasal, intrabronchial, oral or intradermal, or may be any other well-known mode of administration. The administration may further be carried out in a suitable dosage form, for example as a tablet, capsule, powder, granule, suppository, injection, cream or aerosol. Appropriate routes of administration and dosage forms and intervals can be specifically selected depending on the disease or condition to be treated. The skilled person is aware that the type of dosage depends on various factors, such as e.g., the height or body weight, the body surface area, the age, the gender and/or the general health of the patient, but also on the specific agent to be administered, the duration and type of administration, and other medications that may be administered concomitantly.

In a preferred embodiment, the composition according to the invention, as well as possible other additives and/or other active ingredients (e.g., heparin) as defined above, is contained in banked blood (i.e., in whole blood, serum or plasma) and is to be administered together with said banked blood.

Preferred dosage amounts, routes, and forms, as well as preferred administration intervals, are specifically defined below.

In a second aspect, the present invention relates to the use of a composition comprising α-KG and 5-HMF as a dietary supplement for enhancement of hemoglobin-mediated oxygen supply.

The term “dietary supplement”, alternatively also referred to as “supplement”, is well known and comprises such preparations that can be added to human or animal nutrition. The term therefore expressly also includes nutritional supplements for use as animal food.

It is understood that according to the use of the composition according to the invention, comprising α-KG and 5-HMF as dietary supplements, in accordance with the second aspect of the invention described herein, exclusively non-medical (i.e., non-therapeutic) uses are comprised. Thus, the composition may also contain further additives that are beneficial for the intended use, as described in connection with the pharmaceutical composition according to the first aspect of the invention, but to the exclusion of pharmaceutically active ingredients. It is well known that cells require oxygen to produce adenosine triphosphate (ATP), the metabolic energy source for all body functions. In case of insufficient oxygen supply, ATP production comes to a standstill, which can lead to exhaustion and associated loss of performance or a decrease in performance. With increased physical and/or emotional/mental stress, there is an increased need for ATP, and therefore also for oxygen, which is required for ATP production.

Thus, it is assumed by the inventors, without wishing to be bound to a theory, that the provision of the nutrient combination 5-HMF and α-KG according to the use of the invention leads to an increased hemoglobin-mediated oxygen supply in the organism and that this can contribute to maintenance and/or enhancement of ATP production and thus to an improved energy supply. It is assumed that the use of the composition according to the invention, comprising α-KG and 5-HMF as a dietary supplement, is particularly beneficial for preventing and/or reducing performance losses caused by physical and/or mental stress. Thus, it is assumed that the use according to the invention is useful for the enhancement of physical and/or mental performance.

A preferred embodiment according to the second aspect of the invention is directed to the use of the composition as a dietary supplement to enhance physical and/or mental performance, e.g., during sporting activities in competitive or recreational sports or during mental stress, e.g. when studying or at work. The use is particularly preferred to increase aerobic performance and/or endurance performance. Conceivable applications that are expressly contemplated by the invention are the use of the composition as a dietary supplement in endurance sports, in particular in long-distance sports or competitions, such as, e.g., marathon, cycling, swimming, cross-country skiing, as well as in combination sports such as duathlon, triathlon, biathlon etc. Uses of the composition as a dietary supplement for animals, in particular in horse or dog sports, are also being considered.

A further preferred embodiment according to the second aspect of the invention is directed to the use of the composition to reduce, delay or prevent hypoxia-related performance decline.

The term “hypoxia”, also known as “oxygen deficiency” or “hypoxidosis”, refers to an insufficient supply of oxygen affecting the entire body of a living organism or parts of it. Arterial hypoxia is defined as a reduced oxygen partial pressure in the arterial blood and can be indirectly measured by a reduced oxygen saturation of the arterial blood (“hypoxemia”) using known methods (e.g., pulse oximetry).

Hypoxic states/conditions, i.e., states/conditions that result in a hypoxia-related decline in performance can be triggered or promoted by a variety of known factors. For example, hypoxic conditions can occur during intensive sporting activity, in particular if the amount of oxygen taken in through breathing is not sufficient to cover the increased oxygen demand caused by physical activity.

Other possible causes of hypoxic states/conditions are a low oxygen content in the ambient air available for breathing, e.g., due to air pollution from exhaust gases (smog) or fires, as well as low air pressure at high altitudes (e.g., when mountaineering) which is insufficient for the oxygen loading of hemoglobin in the lungs.

Therefore, particularly preferred embodiments according to the second aspect of the invention are directed at the use of the composition as a dietary supplement
(i) to reduce, delay, or prevent hypoxia-related performance decline at high altitudes; and/or
(ii) to enhance physical and/or mental performance at high altitudes; preferably at altitudes above 2000 m, more preferably above 2500 m, even more preferably above 3000 m.

It is to be understood that the altitude stated herein may preferably refer to the altitude in meters (m) above datum line, alternatively to the height in m above sea level (SL).

In a preferred embodiment according to the first or second aspect of the invention, α-KG and 5-HMF are in the (pharmaceutical) composition in a mass ratio of between 1:1 and 100:1, preferably between 2:1 and 50:1, more preferably between 2.25:1 and 25:1.

In the experiments disclosed herein, α-KG and 5-HMF were used in a mass ratio of 3:1. Therefore, in particular, mass ratios of α-KG to 5-HMF in ranges between 2.5:1 and 15:1 are to be understood as particularly preferred, in ranges between 2.75:1 and 10:1 as even more particularly preferred, and in ranges between 3:1 and 5:1 as most preferred. However, it is believed that the observed synergistic effect of the combination of α-KG and 5-HMF can also be achieved with other mass ratios to a comparable, potentially slightly weaker, extent.

In a further preferred embodiment according to the first or second aspect of the invention, the composition is to be administered at a dose of between 5 mg and 500 mg of α-KG per kg body weight and between 5 mg and 160 mg of 5-HMF per kg body weight, preferably at a dose of between 25 mg and 450 mg α-KG per kg body weight and between 8 mg and 150 mg 5-HMF per kg body weight, more preferably at a dose of between 30 mg and 430 mg α-KG per kg body weight and between 10 mg and 145 mg of 5-HMF per kg body weight.

In an alternative preferred embodiment according to the first or second aspect of the invention, the composition is to be administered at a dose of 30-420 mg α-KG per kg body weight and 10-140 mg 5-HMF per kg body weight, more preferably at a dose of 36-300 mg of α-KG per kg body weight and 12-100 mg of 5-HMF per kg body weight, and even more preferably at a dose of 36-130 mg of α-KG per kg of body weight and 12-43 mg of 5-HMF per kg body weight.

It is understood that the preferred doses mentioned can alternatively also be expressed as mg/ml blood volume, assuming that there is approximately 5000 ml of blood volume for a body weight of 70 kg. For example, a dose of 5 mg of 5-HMF per kg body weight can alternatively be expressed as a dose of 0.07 mg of 5-HMF per 1 ml blood volume.

In the experiments disclosed herein, three different dosages were tested for α-KG and 5-HMF, as individual substances and as a combination.

A “low dosage” of 0.42 mg α-KG and/or 0.14 mg 5-HMF per ml whole blood sample (assuming an average of 5000 ml blood volume per 70 kg body weight, this corresponds to about 30 mg α-KG or about 10 mg 5-HMF per kg body weight); a “medium dosage” of 1.8 mg α-KG and/or 0.6 mg 5-HMF per ml whole blood sample (assuming an average of 5000 ml blood volume per 70 kg body weight, this corresponds to about 129 mg α-KG or about 43 mg 5-HMF per kg body weight), and a “high dosage” of 6 mg α-KG and/or 2 mg 5-HMF per ml whole blood sample (assuming an average of 5000 ml blood volume per 70 kg body weight, this corresponds to about 429 mg α-KG or about 143 mg 5-HMF per kg body weight).

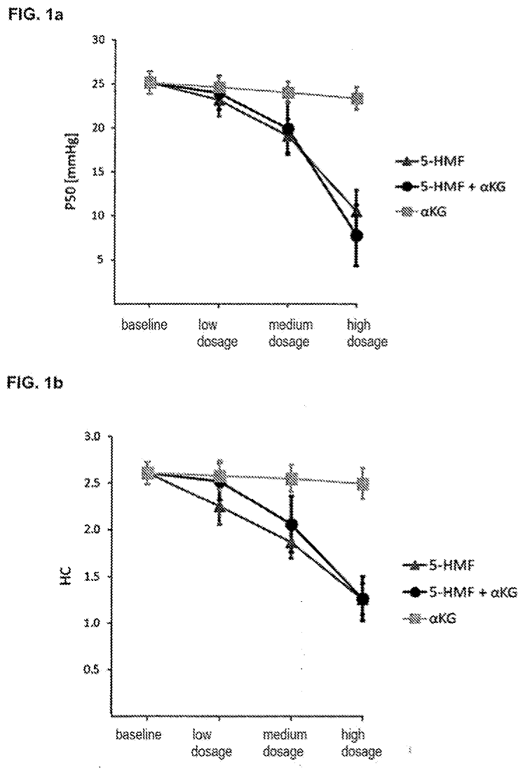

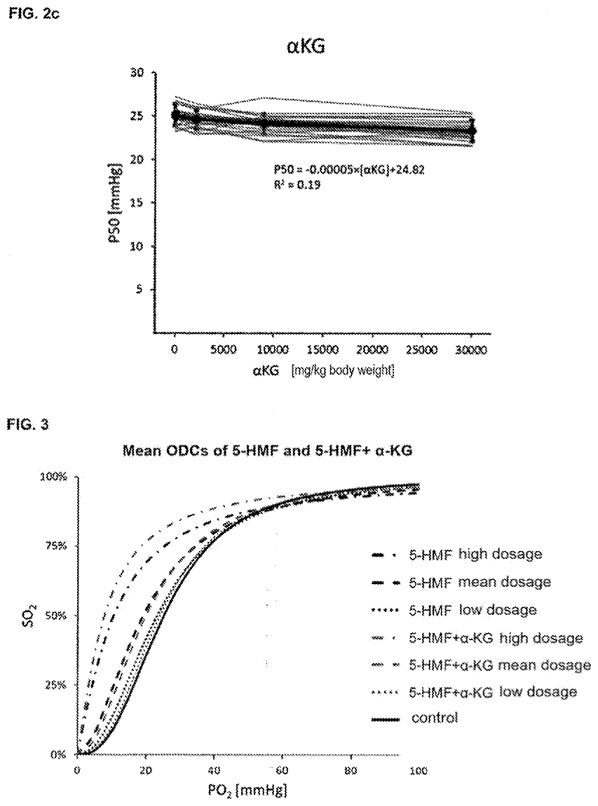

For all three tested dosages of the combination of α-KG and 5-HMF, as well as for 5-HMF alone, the experiments disclosed herein show a significant reduction in the P50 (i.e. the oxygen partial pressure at which 50% of the Hb is saturated with oxygen) from which an enhancement in the Hb oxygen binding affinity can be concluded (see reduction in P50 in FIG. 1a; also clear from the leftward shift of the corresponding ODCs in FIG. 3).

The experimental data also show that the combination of α-KG and 5-HMF, in particular at low and medium doses, results in a comparable increase in Hb oxygen binding affinity compared to 5-HMF alone (FIG. 1a), but in a smaller decrease in the Hill coefficient (as a measure of the cooperativity of Hb oxygen binding) (FIG. 1b). It can be concluded that the additional presence of α-KG preserves the cooperative nature of Hb oxygen binding to a greater extent than with 5-HMF alone (as is clear from the determined Hill coefficient in FIG. 1b; as well as from the preservation of the sigmoid curve shape of the ODC in FIG. 3).

It is therefore assumed that the combination of α-KG and 5-HMF advantageously both improves the oxygen loading of hemoglobin and enables efficient oxygen discharge in the target tissue.

It is understood that the terms “dose” and “dosage” can be used interchangeably in the scope of the present disclosure.

In a further preferred embodiment according to the first or second aspect of the invention, the composition is to be administered as follows: (a) daily, as a single dose or divided into two or more equal or different doses; and/or (b) for at least 2, 3, 4, 5, 6, or 7 consecutive days; and/or (c) 12 hours or less before exposure to ischemic and/or hypoxic conditions.

In a further preferred embodiment according to the first or second aspect of the invention, the composition is to be administered parenterally or orally.

However, in alternative embodiments, the forms, routes, duration and intervals of administration can be adapted individually.

In a preferred embodiment of the first aspect of the invention, the disease or condition is selected from:
(a) a condition characterized by:
   (i) impaired oxygen uptake in the lungs;
   (ii) impaired oxygen diffusion across the air-blood barrier;
   (iii) impaired oxygen loading of hemoglobin;
   (iv) reduced oxygen-carrying capacity of hemoglobin;
   (v) impaired oxygen transport due to restricted blood flow;
   (vi) impaired microvascular oxygen saturation; and/or
   (vii) insufficient oxygen supply;
(b) hypoxemia or anoxemia;
   (c) hypoxia or anoxia;
   (d) ischemia, preferably selected from a gastrointestinal ischemia, cardiac ischemia, cerebral ischemia, renal ischemia, limb ischemia, neuronal ischemia, hypoxic-ischemic brain injury (hypoxic-ischemic encephalopathy), and ischemic neuropathy;
   (e) a respiratory disease, preferably selected from an inflammatory respiratory disease, a respiratory disease associated with a fungal, viral, or bacterial infection, an autoimmune-mediated respiratory disease, an idiopathic respiratory disease, a hyperproliferative respiratory disease, cancer, asthma, chronic obstructive pulmonary disease (COPD), bronchitis, emphysema, pulmonary edema, acute respiratory distress syndrome (ARDS), bronchopulmonary dysplasia (BPD), pulmonary fibrosis, atelectasis, tuberculosis, pneumonia, pneumonitis, sinusitis, allergic rhinitis, pharyngitis, mucositis, stomatitis, bronchiectasis, lupus pneumonitis, cystic fibrosis, and respiratory failure;
   (f) mountain sickness, preferably selected from acute mountain sickness (AMS), high-altitude cerebral edema (HACE), high-altitude pulmonary edema (HAPE), Da Costa syndrome, and chronic mountain sickness (CMS);
   (g) COVID and/or one or more late/post complications thereof, preferably selected from long-COVID and/or post-COVID syndrome; and/or
   (h) chronic fatigue syndrome (CFS).

The term “hypoxemia” refers to a lowered oxygen content (lack of oxygen) in the arterial blood.

The term “anoxemia” refers to “hypoxemia” in its most severe form, i.e. with a significantly reduced oxygen saturation in the blood (far below the physiologically necessary threshold). The term “anoxia” refers to the complete absence of oxygen.

The term “ischemia” is a decrease in blood flow, often associated with pain, or a complete loss of blood flow to a tissue, body part or organ, which can lead to dysfunction. The most common cause of ischemia is a change in blood vessels in the form of a narrowing or occlusion. These can occur, for example, in the case of thrombosis or embolism. The narrowing is referred to as stenosis, for example in atherosclerosis and artery occlusive disease (AOD). Functional narrowing may also occur, such as with Raynaud's syndrome or as a physiological reaction to circulatory shock. Ischemia hinders or stops cellular metabolism. Ischemia caused by restriction or interruption of blood flow is accompanied by a lack of oxygen in the affected area. If nerve tissue is undersupplied for a longer time, a cascade occurs in which high intracellular calcium concentrations contribute to the uncontrolled release of the neurotransmitter glutamate and ultimately damage surrounding tissue cells. These processes can lead to the death of cells (necrosis) and thus to an infarction, e.g. in the case of ischemic heart disease, in which part of the heart muscle does not receive sufficient blood supply, leading to a heart attack. Pressure-related ischemia with tissue damage leads to decubitus (pressure ulcers).

In a preferred embodiment of the first aspect of the invention, hypoxemia is caused by: (a) ventilation-perfusion mismatch; (b) oxygen diffusion disorder; (c) alveolar hypoventilation; (d) right-left shunt; and/or (e) diffusion-perfusion disorder.

In a preferred embodiment of the first aspect of the invention, ischemia is caused by cardiac arrest, shock, carotid occlusion, hypotension, atherosclerosis, asphyxia, thoracic outlet syndrome, hypoglycemia, tachycardia, radiation therapy, chemotherapy, septic shock, heart failure, superior mesenteric artery syndrome, sickle cell disease, thalassemia, induced g-forces, extreme cold, increased stimulation of glutamate receptors, arteriovenous malformations, peripheral arterial occlusive disease, compression or rupture of a blood vessel supplying a tissue or organ, anemia and/or unconsciousness.

According to a preferred embodiment of the first aspect of the invention, the respiratory disease is characterized by reduced oxygen uptake in the lungs caused by a viral infection, wherein the virus underlying the viral infection is preferably a coronavirus, preferably a severe acute respiratory syndrome coronavirus type 2 (SARS-COV-2), or a variant thereof.

“Chronic fatigue syndrome (CSF)” also known as “myalgic encephalomyelitis (ME)” or “ME/CFS” is a chronic disease with the main symptom being exceptionally rapid physical and mental exhaustion, which in extreme cases can lead to extensive disability and the need for care. Despite unexplained causes and mechanisms of development, the syndrome is internationally recognized as an independent disease pattern, with dysregulation of the nervous system, the immune system and/or the endocrine system being observed. Studies have shown that CFS patients usually show a pronounced metabolic disorder in the energy balance of the somatic cells, with the ATP synthesis that is provided by the mitochondria being restricted. As a result, the body chronically has too little energy available. This energy deficit is caused, among other things, by nitrosative stress with high levels of nitrogen monoxide (NO). Due to the inhibition of the mitochondrial respiratory chain (synthesis of ATP), cells with a high energy requirement are primarily affected: muscles, neuronal cells, immune system, heart muscle. In view of the effect of the composition according to the invention as determined in the context of the present invention, it can be assumed that the energy deficit and associated disease symptoms (in particular exhaustion/fatigue), which is typically present in CFS patients, can be therapeutically counteracted by the inventive use of the pharmaceutical composition according to the first aspect of the invention. Thus, it can, in particular, be assumed that the increased supply of oxygen in the peripheral tissue has a beneficial effect on mitochondrial energy production (i.e. ATP synthesis), and that, thus, the states of exhaustion associated with these diseases, which, apart from lack of oxygen supply, are also caused by an impaired mitochondrial function (mitochondrial dysfunction and/or impaired ATP synthesis), can be at least partially counteracted.

The terms “long Covid”, also referred to as “post-COVID/long-COVID” or “post-/long-COVID”, “long/post-COVID”, “post-COVID”, “post-COVID syndrome” and “post-COVID-19 condition according to ICD-10 GM”, in English also: “post-acute sequelae of COVID-19 (PASC)”, “chronic COVID syndrome (CCS)”, “COVID-19 long-hauler”, or “post-acute Covid-19 syndrome” refer to the late or long-term health consequences of the Coronavirus disease 2019 (COVID-19). As a rule, the acute COVID-19 infection lasts up to four weeks, but it can last for several months, for example if inpatient treatment in an intensive care unit is required. However, longer-term symptoms can persist beyond this period or occur additionally, even with a milder course of the disease or an undetected infection. In very rare cases, symptoms of long COVID also occur as a result of a vaccination against the virus (post vaccine syndrome). There is no single, common definition for long-term consequences so far. Symptoms observed include severe lung damage, inflammatory reactions and changes in various organs, including shortness of breath, fatigue (post-COVID tiredness), clouded consciousness and neurological disorders. The most commonly reported symptoms include, among others, respiratory problems, neurological disorders, mental health impairments, restricted mobility, fatigue and muscle weakness. “long-COVID” and “post COVID” (also referred to interchangeably as “post-COVID syndrome”) can be similar in symptoms and are generally differentiated according to the period in which the symptoms have existed into long-COVID (continuation of, or new onset of symptoms(s) longer than 4 weeks after the acute infection) and post-COVID syndrome (symptoms persisting or emerging for more than 12 weeks).

In a similar way to CFS, the use of the pharmaceutical composition according to the first aspect of the invention and the therewith associated enhancement in hemoglobin-mediated oxygen delivery can therefore also be expected to have positive therapeutic and/or preventive effects not only in the treatment of an acute COVID disease, but also in the treatment and/or prevention of “long COVID” and/or the so-called “post-COVID syndrome”, in particular in the treatment and/or prevention of associated fatigue states.

In the context of the present invention, the term “COVID” generally refers to coronavirus disease caused by an infection with severe acute respiratory syndrome coronavirus 2 (SARS-CoV-2) or a variant thereof (corona virus disease), preferably Corona Virus Disease-19 (COVID-19)).

In a particularly preferred embodiment of the above embodiment (g), the late/post-complication of COVID (e.g., long-COVID and/or post-COVID syndrome) is characterized by one or more of the following symptoms:
physical and/or mental exhaustion, preferably triggered by reduced oxygen uptake capacity of the lungs and/or peripheral tissue; and/or
reduced/impaired oxygen transport capacity and/or reduced/impaired loading of hemoglobin with oxygen; and/or
reduced/impaired mitochondrial energy production (ATP synthesis).

In a third aspect, the present invention relates to an in vitro method for enhancing the hemoglobin-mediated oxygen delivery capacity of blood, comprising mixing a blood sample with a composition as defined in the context of the first or the second aspect of the present invention.

The term “hemoglobin-mediated oxygen delivery capacity of blood” as used herein is interchangeable with the term “hemoglobin-mediated oxygen supply capacity of blood” and refers to the ability of red blood cells (erythrocytes) contained in the blood to deliver or supply oxygen to tissues, organs and/or cells based on the reversible oxygen binding to hemoglobin.

In a preferred embodiment, the method comprises
(i) contacting a blood sample, preferably a whole blood sample, comprising erythrocytes with the composition according to the invention under atmospheric conditions that promote oxygen loading over oxygen discharge of the hemoglobin contained in the erythrocytes; and
(ii) maintaining the blood sample under the atmospheric conditions until further use of the blood sample.

In a preferred embodiment, the atmospheric conditions are characterized by an oxygen partial pressure of at least 30 mmHg.

In a fourth aspect, the present invention relates to an in vitro method for reducing or preventing hypoxia-induced damage to an organ or tissue, comprising:
(a) contacting an isolated organ or tissue in vitro with a preservation solution, wherein the preservation solution comprises the composition as defined in connection with the first or second aspect of the invention; and
(b) maintaining the organ or tissue in vitro in contact with the preservation solution until further use of the organ or tissue;

wherein preferably:
(i) the contacting of the organ or tissue with the preservation solution corresponds to a complete immersion of the organ or tissue into the preservation solution; and/or
(ii) the preservation solution further comprises erythrocytes, preferably plasma or whole blood.

In a preferred embodiment of the method according to the third or fourth aspect of the invention, α-KG and 5-HMF are to be applied at a concentration of
(i) between 0.07 mg and 7 mg α-KG and between 0.07 mg and 2.24 mg 5-HMF per 1 ml of the blood sample or the preservation solution; or
(ii) between 0.005 mg and 0.5 mg α-KG and between 0.005 mg and 0.160 mg 5-HMF per
1 g of the organ or tissue,

preferably:
(i) between 0.35 mg and 6.3 mg α-KG and between 0.11 mg and 2.1 mg 5-HMF per 1 ml of the blood sample or the preservation solution; or
(ii) between 0.025 mg and 0.45 mg α-KG and between 0.008 mg and 0.150 mg 5-HMF per 1 g of the organ or tissue,

more preferably:
(i) between 0.42 mg and 6 mg α-KG and between 0.14 mg and 2.03 mg 5-HMF per 1 ml of the blood sample or the preservation solution; or
(ii) between 0.03 mg and 0.43 mg α-KG and between 0.01 mg and 0.145 mg 5-HMF per 1 g of the organ or tissue.

In an alternative preferred embodiment of the method according to the third or fourth aspect of the invention, α-KG and 5-HMF are to be applied at a concentration of
(i) 0.42-5.88 mg α-KG and 0.14-1.96 mg 5-HMF per 1 ml of the blood sample or the preservation solution; or
(ii) 0.03-0.42 mg α-KG and 0.01-0.14 mg 5-HMF per 1 g of the organ or tissue; more preferably:
(i) 0.50-4.20 mg α-KG and 0.17-1.4 mg 5-HMF per 1 ml of the blood sample or the preservation solution; or
(ii) 0.04-0.30 mg α-KG and 0.01-0.10 mg 5-HMF per 1 g of the organ or tissue; even more preferably:
(i) 0.5-1.8 mg α-KG and 0.2-0.6 mg 5-HMF per 1 ml of the blood sample or the preservation solution; or
(ii) 0.04-0.13 mg α-KG and 0.01 and 0.04 mg 5-HMF per 1 g of the organ or tissue.

In a fifth aspect, the present invention relates to in vitro use of a composition as defined in the context of the first and second aspect of the present invention, for enhancing hemoglobin-mediated oxygen supply to an organ or tissue.

In a preferred embodiment of the use according to the fifth aspect of the invention:
(i) the organ or tissue is an organ or tissue isolated from a human or animal body; and/or
(ii) the composition is contained in a preservation solution (as defined in the context of the fourth aspect of the invention) and said composition is to be contacted with the organ or the tissue in such a way that the organ or the tissue is completely immersed into the preservation solution.

In a preferred embodiment of the method according to the fourth aspect of the invention or the use according to the fifth aspect of the invention, the organ or the tissue is a transplant; wherein the transplant is preferably selected from:
(i) kidney, liver, heart, pancreas, lung, skin, subcutaneous tissue, muscle and/or bone; and or
(ii) a free flap surgery, preferably a free skin, skin-muscle, and/or skin-muscle-bone flap surgery.

In a sixth aspect, the present invention relates to oxygen-enriched banked blood which is produced using the method according to the third aspect of the invention.

In a seventh aspect, the present invention relates to a method for treating and/or preventing a disease or condition that benefits from enhanced hemoglobin-mediated oxygen delivery, wherein a pharmaceutical composition comprising α-ketoglutaric acid (α-KG) and 5-hydroxymethyl-2-furfural (5-HMF) is to be administered at a pharmaceutically effective dose to an individual suffering from or at risk of developing such disease or condition or of being exposed to such disease or condition.

It is understood that the embodiments and preferred embodiments disclosed in connection with the first aspect of the invention, in particular those relating to diseases or conditions to be treated, relating to the dosage and administration of the composition of the invention as well as possible additives, if applicable, also apply to the method according to the seventh aspect of the invention.

The term “oxygen saturation (SO₂)” refers to the quotient of oxygen present in the blood and the maximum oxygen capacity of the blood in percent.

The term “peripheral oxygen saturation (SpO₂)” refers to the oxygen saturation in a tissue and can be determined using various measuring methods known to those skilled in the art, e.g. by non-invasive pulse oximetry.

The term “inspiratory oxygen fraction” (“FiO₂” abbreviated from: “fraction of inspired oxygen”) is the proportion of oxygen in the inhaled air.

The term “maximum oxygen uptake” (abbreviated “VO₂max”) indicates the maximum number of milliliters of oxygen the body can use per minute when under exercise and is generally given in milliliters of oxygen per minute (ml O₂/min). VO₂max can be used as a criterion for evaluating a person's endurance performance.

In an eighth aspect, the present invention relates to a pharmaceutical composition comprising α-ketoglutaric acid (α-KG) and 5-hydroxymethyl-2-furfural (5-HMF):
(a) to enhance hemoglobin-mediated oxygen supply to mitochondria; and/or
(b) to enhance mitochondrial energy production (preferably, mitochondrial ATP synthesis);

for use in the treatment and/or prevention of a disease or condition that benefits from one or more of the effects mentioned in (a) and (b).

In connection with the eighth aspect of the present invention, it is to be understood that the disease or condition can preferably be selected from one or more of the diseases or conditions disclosed in connection with the first aspect of the present invention. It should also be understood that each of the preferred embodiments disclosed in connection with the first aspect of the invention, in particular with regard to the mixing ratios of the substances present in the composition, as well as the possible dosages and formulations and dosage forms/types, if applicable, can also apply to the pharmaceutical composition according to the eighth aspect of the invention and should be understood as being directly disclosed in connection with the latter.

Unless otherwise defined, all technical and scientific terms used herein have the same meaning as generally understood by one skilled in the art to which this invention pertains. In the event of a conflict, the patent specification, including definitions, will prevail.

It is understood that the definitions and embodiments of the invention described above in connection with the first aspect of the present invention also apply accordingly to the second, third, fourth and any further aspects of the present invention, where possible.

With respect to the embodiments described in this specification, particularly in the claims, it is intended that any embodiment mentioned in a dependent claim may be combined with any embodiment of any claim (independent or dependent) on which that dependent claim depends. In the case of an independent claim 1 specifying 3 alternatives A, B and C, a dependent claim 2 specifying 3 alternatives D, E and F, and a claim 3 dependent on claims 1 and 2 and specifying 3 alternatives G, H and I, it is to be understood, for example, that the description clearly discloses embodiments corresponding to the combinations A, D, G; A, D, H; A, D, I; A, E, G; A, E. H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I unless expressly stated otherwise. This also applies to the combination of different alternatives that are mentioned in different subclaims.

Similarly, and even in cases where no alternatives are recited in independent and/or dependent claims, if dependent claims refer to a plurality of preceding claims, any combination of subject matter covered by those claims is deemed to be expressly disclosed. For example, in the case of an independent claim 1, a dependent claim 2 which refers back to claim 1 and a dependent claim 3 which refers back to both claim 2 and claim 1, the combination of the subject matter of claims 3 and 1 is, for example, equally directly and unambiguously disclosed as the combination of the subject matter of claims 3, 2 and 1. If there is a further dependent claim 4 relating to one of claims 1 to 3, it follows that the combination of the subject matter of claims 4 and 1, claims 4, 2 and 1, claims 4, 3 and 1 as well of claims 4, 3, 2 and 1 is directly and unambiguously disclosed.

The invention is described herein, by way of example only, with reference to the accompanying figures and for the purpose of illustrating the preferred embodiments of the present invention.

The figures show:

FIG. 1: Effects of the substances in different dosages (mean±SD). a) Mean P50 of all subjects at baseline and different dosages. b) Mean HC value of all subjects at baseline and different dosages.

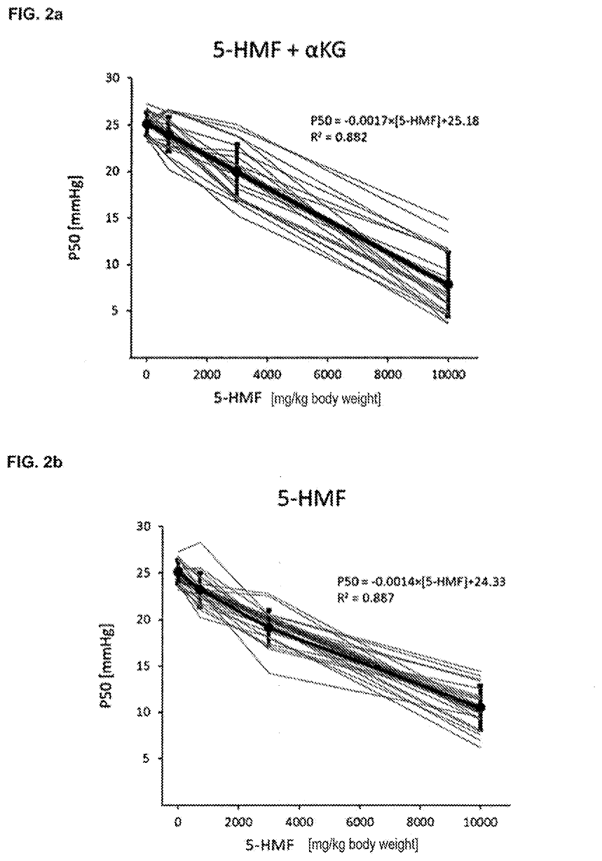

FIG. 2: Dose dependence of P50 changes. Light gray lines show individual P50 values, the black line corresponds to P50 values of all subjects (mean±SD), and the dark gray line is the linear regression function the characterization of which (P50 and R² values) is given in the graph. a) Dose dependence of 5-HMF+α-KG. b) Dose dependence of 5-HMF. c) Dose dependence of α-KG.

FIG. 3: Mean ODC of 5-HMF (black) and 5-HMF+α-KG (gray), represented by the interaction of mean P50 and mean HC of all subjects.

The examples illustrate the invention.

### EXAMPLES

### Example 1: Brief Summary of the Experiments Shown in Examples 2 to 4

The present study deals with the investigation of possible effects of the active ingredients 5-HMF, α-KG, as well as of the combination of 5-HMF and α-KG on the oxygen binding curve of hemoglobin. Oxygen binding curves were determined using a novel in vitro method (described in Ref: 20: Woyke S, Ströhle M, Brugger H, Strapazzon G, Gatterer H, Mair N, Haller T. High-throughput determination of oxygen dissociation curves in a microplate reader—A novel, quantitative approach. Physiol Rep. 2021 Aug;9(16):e14995. doi: 10.14814/phy2.14995. PMID: 34427400) on human whole blood samples. Three different dosages (low, medium, and high) were used. A strong dose-dependent increase in Hb-O₂ affinity was noted for 5-HMF alone and the combined addition of 5-HMF and α-KG. α-KG alone also increased Hb-O₂ affinity, but to a lesser extent. With increasing Hb-O₂ affinity, the sigmoid shape of the oxygen binding curve was better preserved by the combination of 5-HMF and α-KG than by 5-HMF alone. This effect, observed for the first time in the present experiments, suggests a previously unknown synergism of the combination of α-KG and 5-HMF regarding the allosteric regulation of hemoglobin, which is probably due to α-KG-mediated stabilization of 5-HMF, which is known to be highly reactive.

### Example 2: Materials & Methods

Venous blood was collected from 20 healthy subjects (ten women, ten men) aged 18 to 40 years. The subjects were nonsmokers, not pregnant or breastfeeding, had no known hemoglobinopathy, and did not have a recent history of illness, trauma, blood loss, or multi-day travel to high altitudes (>3000 m). Immediately after blood collection, blood gas analysis was performed (ABL800 flex, radiometer) and samples were stored on ice and processed within eight hours.

5-HMF and α-KG were provided in different carrier solutions (aqua, glucose, glucose and phosphoric acid, acid, aqua and sodium hydroxide, aqua and phosphoric acid, and glucose and sodium hydroxide), and control solutions consisted of the carrier solutions alone without 5-HMF or α-KG. Ten microliters of three different concentrations each of 5-HMF or α-KG and 5-HMF plus α-KG were added to 100 μl of whole blood and mixed gently by resuspension.

The recommended maximum daily dose of 5-HMF of Sanopal® (Cyl, Austria) is 720 mg. Assuming its total distribution in the blood volume of a 70 kg person to be approximately 5000 ml (16), the low dose of 5-HMF was defined as 0.14 mg/ml (700 mg/70 kg). The medium dose of 5-HMF (0.6 mg/ml, 3,000 mg/70 kg) was chosen to correspond to a usual dosage of Aes-103, a known anti-sickle cell agent (10) containing 5-HMF. The high dose of 5-HMF (2 mg/ml, 10,000 mg/70 kg) corresponds to a dosage of 5-HMF (143 mg/kg) which has been shown in animal studies to produce adverse effects such as an increase in serum gamma globulin levels and the relative spleen weight (17, 18). α-KG was used in the low dose with 0.42 mg/ml (2,100 mg/70 kg), in the medium dose with 1.8 mg/ml (9,000 mg/70 kg), and in the high dose with 6 mg/ml (30,000 mg/70 kg). This means that when the two active ingredients α-KG and 5-HMF were used in combination, they were present in a mass ratio of 3:1 (α-KG:5-HMF).

### ODC Measurements

For each of the three described test concentrations of the active ingredients alone (α-KG or 5-HMF) or of their combination (α-KG:5-HMF), ODCs (as duplicate measurements) were determined using the in vitro measurement method for high-throughput determination of oxygen dissociation curves (ODC) described by Woyke et al. (20), that is meanwhile patent-pending (Ref. 20: Woyke, S., Ströhle, M., Brugger, H., Strapazzon, G., Gatterer, H., Mair, N., & Haller, T. (2021). High-throughput determination of oxygen dissociation curves in a microplate reader—A novel, quantitative approach. Physiological Reports, 9, e14995. https://doi.org/10.14814/phy2.14995); see also “ASCENION GmbH—Novel gas flow system for high throughput determination of oxygen dissociation curves (ODC)” at: https://www.ascenion.de/en/technology-offers/novel-gas-flow-system-for-high-throughput-determination-of-oxygen-dissociation-curves-odc.

This real-time, high-throughput analysis enabled, for the first time, precise and efficient ODC determination in whole blood samples using a simple adaptation of standard microplate readers and specially modified (gas-perfused) 96-well microliter measuring plates. To ensure stable conditions during the ODC measurement, the measurement setup was set up in an environmentally controlled, closed device (EC box). This box is temperature-controlled (37° C.) and contains the experimental setup consisting of a humidifier, a cover for the microplate reader, tubes and valves. A modified 96-well plate was used (a special coating process ensures a thin, yet adherent layer of red blood cells for optimal fluorescence measurement), which enabled a constant gas flow across all wells in a meandering gas flow channel. The measuring plate is integrated into a gas system consisting of gas bags, gas hoses, humidifiers, gas mixers and a peristaltic pump.

The blood samples were placed in the individual wells using a special stamping technique so that a film consisting of a few cell layers could form. Oxygen saturation was measured in each well using dual-wavelength spectroscopy and oxygen partial pressure was measured using the fluorescence lifetime of commercial oxygen sensors at the inlet and outlet ports of the measuring plate. An oxygen ramp of around 20% by volume to 0% by volume was formed, so that a dissociation curve can be measured. The measurements were carried out every minute in a conventional fluorescence plate reader, so that up to 92 samples of whole blood could be analyzed within ˜25 min.

For the ODC experiments, gas mixtures containing 40 mmHg PCO₂ were used, and the temperature was kept constant at 37° C.

### Data Processing and Statistical Analysis:

Since the statistical analysis showed no significant differences with respect to the carrier solution, the results of the different carrier solutions per concentration level were averaged for the final analysis.

Curve fits, P₅₀ and Hill coefficient (HC) calculations, and graphs were created using Excel (Microsoft 2016). The statistical analysis was performed using IBM SPSS Statistics 25. The unpaired t-test was used to analyze baseline differences between substance compositions. ANOVA with repeated measures design was used to determine P₅₀ and HC differences between the different dosages and substance compositions.

Post-hoc t-test (Bonferroni-corrected) and ANOVA with repeated measures design were used for each individual substance or a combination of two substances to identify the location and magnitude of changes. Linear regression analysis was used to determine the concentration dependence for each substance. P<0.05 was considered significant. Data are presented as mean±SD.

### Example 3: Results

The average age of the subjects was 29.6±3.0 years. The hemoglobin concentration (14.5±1.3 g/dl) and hematocrit (44.4±4.0%) were normal in this population, while the pH was slightly lower than 7.40 (7.35±0.04), corresponding to the higher carbon dioxide levels (48.4±7.4 mmHg) in venous blood compared to arterial blood.

At the start of the study, the P50 values were 25.1±1.3 and showed a slight gender-specific difference (women 26.0±1.0 mmHg vs. men 24.3±0.9 mmHg; P=0.001), and the HC values were 2.61±0.21.

The ANOVA with repeated measures design showed significant total substance (P<0.001), dose (P<0.001) and interaction (substance×dose, P<0.001) effects for both P50 and HC (FIG. 1).

5-HMF significantly changed P50 (P<0.001) (FIGS. 1 and 2) and HC (P<0.001) (FIG. 1b) in a dose-dependent manner. α-KG alone also altered P50 (P<0.001) and HC (P=0.012), but numerically compared to 5-HMF to a significantly lesser extent (FIG. 1).

The addition of α-KG to 5-HMF showed a general dose and substance effect for P50 and HC (P<0.001) compared to 5-HMF alone and specifically on P50 at the highest dose (P=0.028) (FIG. 1a) and on HC at the lowest and medium doses (P<0.001 and P=0.015) (FIG. 1b).

For all substances and combinations, the dose dependence of P50 followed a linear course (FIG. 2).

Overall, changes in P50 and HC alter the shape of the ODC in a specific way (FIG. 3). At low doses and for the control solution, the typical sigmoid shape of the ODC is clearly apparent. At medium doses, the sigmoid character is preserved, but due to the higher HC, the ODC for the combination 5-HMF+α-KG has a more preserved sigmoid shape compared to 5-HMF alone. At high doses, both ODCs lost their typical sigmoid shape due to the very low P50 and HC values.

### Example 4: Discussion

The most important finding of the present study is that the combined administration of 5-HMF and α-KG increased HbO₂ affinity in human whole blood. A linear dose dependence was found for all substances and combinations, but to different extents (i.e., α-KG shows the lowest effect).

Interestingly, HC differed between the substance combinations, particularly at low and medium doses, indicating changes in the shape of the ODC in addition to the P50-related shifts (FIG. 3).

A notable finding of the present study is the observed combined effect of 5-HMF and α-KG on the Hill coefficient (HC). Changes in the HC value directly influence the sigmoid shape of the ODC (2, 19), which is crucial for the oxygen transport of hemoglobin. A steeper increase of the ODC facilitates hemoglobin oxygen transport (2) because smaller changes in PO₂ induce larger changes in SpO₂, implying that O₂ loading at lung level, as well as O₂ unloading at tissue level, is facilitated.

As shown by the data disclosed herein, the combination of 5-HMF with α-KG, particularly with low and medium doses, can maintain the sigmoid curve shape of the ODC while increasing HbO₂ affinity compared to 5-HMF alone (FIG. 3).

This effect, observed for the first time in the present experiments, suggests a previously unknown synergism of the combination of α-KG and 5-HMF regarding the allosteric regulation of hemoglobin, which is probably due to α-KG-mediated stabilization of 5-HMF, known to be highly reactive.

### CONCLUSION

5-HMF induces a strong dose-dependent increase in HbO₂ affinity. The combination of 5-HMF with α-KG shows higher HC values at lower P50 compared to 5-HMF alone, thereby preserving the sigmoid shape of the oxygen binding curve. As a result, efficient oxygen transport, i.e. the oxygen supply to the tissue, can be maintained.

### Example 5: Exemplary Test Procedures

The physiological effect of the composition of the invention on which the present invention is based becomes manifest, among other things, in a measurable increase in physical performance (strength and/or endurance) compared to corresponding control individuals who were administered placebos. Exemplary evaluation methods that are known in the state of the art and can be routinely used for these and similar purposes are, without any intended limitation, muscle strength measurement (e.g., arm strength measurement or respiratory muscle strength measurement), the so-called 1-minute sit-to-stand test, comparing the running/walking distance covered in a certain time (e.g., in the form of a so-called 6-minute walking test (also known as “6MWT”). In addition, the effects observed in the in vitro test methods disclosed herein (see Examples 1 to 4) can also be evaluated using other common direct and/or indirect measurement methods for determining arterial oxygen saturation (qualitative and/or quantitative), with exemplary methods including, in particular, pulse oximetry measurement, blood gas analysis (e.g., using spiroergometry), heart rate variability analysis (HRV analysis), and near-infrared spectroscopy (NIRS). Various commercial test assays are also available that can be used to measure and quantify ATP levels in samples obtained from patients/test individuals in vitro (e.g., Luminescent ATP Detection Assay Kit by abcam (www.abcam.com)). Various clinical studies that additionally demonstrate the above-mentioned effects (in particular also directly on humans) are being planned or are about to be implemented at the time of filing this patent application.

It is understood that the examples and embodiments described herein are for illustrative purposes only and that various modifications and changes to the examples and embodiments as well as combinations of features otherwise described in this application will be readily apparent to a person skilled in the art and are included in the disclosure content of the invention described herein and in the scope of protection of the claims. All patents and patent applications referred to herein are hereby incorporated into the disclosure content of the application.

All patents, patent applications, publications and documents referenced herein are incorporated by reference in their entirety. Reference to the above-mentioned patents, patent applications, publications and documents is neither an admission that any of the foregoing patents, patent applications, publications and documents are relevant prior art, nor does it constitute an admission as to the content or date of such publications or documents. Their mention is not an indication of a search for relevant disclosures. Any statements regarding the date or content of the documents are based on available information and do not constitute an admission of their accuracy or correctness.

Changes may be made to the foregoing without departing from the fundamental aspects of the technology. However, although the technology has been described in substantial detail with reference to one or more specific embodiments, those of skill in the art will recognize that changes may be made to the embodiments specifically disclosed in this application. However, these changes and improvements are within the scope of the present invention.

The technology described herein by way of illustration can be conveniently carried out in the absence of elements not specifically disclosed herein. For example, in each case the terms “comprising”, “consisting of” and “consisting essentially of” and “consisting of” may be replaced by one of the other two terms.

The terms and expressions used are intended to be descriptive rather than limiting, and the use of such terms and expressions does not exclude equivalents of the features or parts thereof shown and described, and various modifications are possible within the scope of the claimed technology. The terms “method” and “procedure” are used interchangeably herein.

The term “a” or “an” may refer to one or more of the elements it modifies (e.g., “a carrier” may mean one or more carriers) unless it is clear from the context that either one of the elements or more than one of the elements is being described.

The term “approximately” or “about” as used herein refers to a value within 10% of the underlying parameter (i.e., plus or minus 10%), and it is understood that use of the term “approximately” or “about” at the beginning of a series of values modifies each of the values (i.e., “approximately 1, 2, and 3” refers to approximately 1, approximately 2, and approximately 3). For example, a weight of “approximately 100 grams” may include weights between 90 grams and 110 grams. If a list of values is described herein (e.g., about 50%, 60%, 70%, 80%, 85% or 86%), the list includes all intermediate values and fractions thereof (e.g., 54%, 85.4%).

Thus, it should be understood that although the present technology has been specifically disclosed through representative embodiments and optional features, modifications and variations of the concepts disclosed herein may be made by those of skill in the art and that such modifications and variations are considered within the scope of this technology.

Certain embodiments of the technology are set out in the following claims.

### FURTHER REFERENCES

1. Mairbaurl H, Weber R E. Oxygen transport by hemoglobin. Compr Physiol. 2012;2(2).1463-89.
2. Imai K. Allosteric effects in haemoglobin. Cambridge, UK: Cambridge University Press; 1982.
3. Böning D, Littschwager A, Hütler M, Beneke R, Staab D. Hemoglobin oxygen affinity in patients with cystic fibrosis. PLoS One. 2014;9(2):e97932.
4. Mairbäurl H, Weber R E. Oxygen transport by hemoglobin. Compr Physiol. 2012;2(2):1463-89.
5. Dempsey J A. With haemoglobin as with politics—should we shift right or left? J Physiol. 2020;598(8):1419-20.
6. Srinivasan A J, Morkane C, Martin D S, Welsby I J. Should modulation of p50 be a therapeutic target in the critically ill? Expert Rev Hematol. 2017;10(5):449-58.
7. Woyke S, Rauch S, Strohle M, Gatterer H. Modulation of Hb-O2 affinity to improve hypoxemia in COVID-19 patients. Clin Nutr. 2020.
8. Yalcin O, Cabrales P. Increased hemoglobin O2 affinity protects during acute hypoxia. Am J Physiol Heart Circ Physiol. 2012;303(3):H271-81.
9. Mahon R T, Carlone G E, Roney N G, Swift J M. Cardiovascular Parameters in a Swine Model of 205 Normobaric Hypoxia Treated With 5-Hydroxymethyl-2-Furfural (5-HMF). Front Physiol. 2019;10;395.
10. Stern W, Matthews D, McKew J C, Shen J, G J K. A phase 1, first-in-man, dose-response study of Aes-103 (5-HMF), an anti-sickling, allosteric modifier of hemoglobin oxygen affinity in healthy normal volunteers. Blood. 2012;120(21):3210.
11. Hannemann A, Cytlak U M, Rees D C, Tewari S, Gibson J S. Effects of 5-hydroxymethyl-2-furfural on the volume and membrane permeability of red blood cells from patients with sickle cell disease. J Physiol. 2014;592(18):4039-49.
12. Kössler F, Mair L, Burlscher M, Gatterer H. 5-Hydroxymethylfurfural and alpha-ketoglutaric acid supplementation increases oxygen saturation during prolonged exercise in normobaric hypoxia. Int J Vitam Nutr Res. 2019:1-6.
13. Matzi V, Lindenmann J, Muench A, Greilberger J, Juan H, Wintersteiger R, et al. The impact of preoperative micronutrient supplementation in lung surgery. A prospective randomized trial of oral supplementation of combined alpha-ketoglutaric acid and 5-hydroxymethylfurfural. Eur J 218 Cardiothorac Surg. 2007;32(5):776-82.
14. Formenti F, Vogel D J, Camporota L. Nutritional interventions to modulate haemoglobin oxygen affinity in COVID-19 patients. Clin Nutr. 2020;39(12):3843-4.
15. Woyke S, Gatterer H, Rauch S, Ströhle M. Reply—Letter to the editor—Nutritional interventions to modulate haemoglobin-oxygen affinity in COVID-19 patients. Clin Nutr. 2020;39(12):3842.
16. Sharma R, Sharma S. Physiology, Blood Volume. StatPearls. Treasure Island (FL): StatPearls Publishing Copyright © 2020, StatPearls Publishing LLC.; 2020.
17. NTP toxicology and carcinogenesis studies of 5-(Hydroxymethyl)-2-furfural (CAS No. 67-47-0) in F344/N rats and B6C3F1 mice (gavage studies), Natl Toxicol Program Tech Rep Ser. 2010(554):7-13, 5-9, 21-31 passim.
18. Zaitsev A N, Simonian T A, Pozdniakov A L. [Hygienic standards for hydroxymethylfurfural in food products]. Vopr Pitan. 1975(1):52-5.
19. Goutelle S, Maurin M, Rougier F, Barbaut X, Bourguignon L, Ducher M, et al. The Hill equation: a review of its capabilities in pharmacological modelling. Fundam Clin Pharmacol. 2008;22(6):633-48.
20. Woyke S, Ströhle M, Brugger H, Strapazzon G, Gatterer H, Mair N, Haller T. High-throughput determination of oxygen dissociation curves in a microplate reader—A novel, quantitative approach. Physiol Rep. 2021 Aug;9(16):e1495. doi: 10.14814/phy2.14995. PMID; 34427400.

1. A method of treating or preventing a disease or condition that benefits from an enhanced hemoglobin-mediated oxygen delivery, comprising administering to a subject a pharmaceutical composition comprising α-ketoglutaric acid (α-KG) and 5-hydroxymethyl-2-furfural (5-HMF).

2. A method of enhancing hemoglobin-mediated oxygen supply, comprising administering to a subject a composition comprising α-ketoglutaric acid (α-KG) and 5-hydroxymethyl-2-furfural (5-HMF).

3. The method of claim 1, wherein the α-KG and the 5-HMF are present in the composition in a mass ratio of between 1:1 and 100:1, preferably between 2:1 and 50:1.

4. The method of claim 1, wherein the composition comprises between 5 mg and 500 mg of the α-KG per kg of the subject's body weight and between 5 mg and 160 mg of the 5-HMF per kg of the subject's body weight.

5. The method of claim 1, comprising administering the composition:
(a) daily, as a single dose or divided into two or more equal or different doses;
(b) for at least 5 consecutive days; or
(c) 12 hours or less before exposure to ischemic or hypoxic conditions.

6. The method of claim 1, wherein the disease or condition is:
(a) a condition characterized by:
(i) impaired oxygen uptake in the lungs;
(ii) impaired oxygen diffusion across the air-blood barrier;
(iii) impaired oxygen loading of hemoglobin;
(iv) reduced oxygen-carrying capacity of hemoglobin;
(v) impaired oxygen transport due to restricted blood flow;
(vi) impaired microvascular oxygen saturation; or
(vii) insufficient oxygen supply;
(b) hypoxemia or anoxemia;
(c) hypoxia or anoxia;
(d) ischemia;
(e) a respiratory disease;
(f) a mountain sickness;
(g) COVID or one or more complications thereof; or
(h) chronic fatigue syndrome (CFS).

7. The method of claim 6, wherein the hypoxemia is caused by:
(a) ventilation-perfusion mismatch;
(b) oxygen diffusion disorder;
(c) alveolar hypoventilation;
(d) right-left shunt; or
(e) diffusion-perfusion disorder.

8. The method of claim 6, wherein the ischemia is caused by cardiac arrest, shock, carotid occlusion, hypotension, atherosclerosis, asphyxia, thoracic outlet syndrome, hypoglycemia, tachycardia, radiation therapy, chemotherapy, septic shock, heart failure, superior mesenteric artery syndrome, sickle cell disease, thalassemia, induced g-forces, extreme cold, increased stimulation of glutamate receptors, arteriovenous malformations, peripheral arterial occlusive disease, compression or rupture of a blood vessel supplying a tissue or organ, anemia or unconsciousness.

9. The method of claim 6, wherein the respiratory disease is characterized by reduced oxygen uptake in the lungs caused by a viral infection.

10. An in vitro method for enhancing the hemoglobin-mediated oxygen delivery capacity of blood, comprising mixing a blood sample with a composition comprising α-ketoglutaric acid (α-KG) and 5-hydroxymethyl-2-furfural (5-HMF).

11. An in vitro method for reducing or preventing hypoxia-induced damage to an organ or tissue, comprising:
(a) contacting an isolated organ or tissue in vitro with a preservation solution, wherein the preservation solution comprises a composition comprising aα-ketoglutaric acid (α-KG) and 5-hydroxymethyl-2-furfural (5-HMF); and
(b) maintaining the organ or tissue in vitro in contact with the preservation solution until further use of the organ or tissue.

12. The method of claim 10, wherein α-KG and 5-HMF are in a concentration of
between 0.07 mg and 7 mg α-KG and between 0.07 mg and 2.24 mg 5-HMF per 1 ml of the blood sample.

13. (canceled)

14. The method of claim 11, wherein the organ or tissue is a transplant.

15. A method of treating or preventing a disease or condition that benefits from (a) an enhanced hemoglobin-mediated oxygen supply to mitochondria or (b) an enhanced mitochondrial energy production, comprising administering to a subject a pharmaceutical composition comprising α-ketoglutaric acid (α-KG) and 5-hydroxymethyl-2-furfural (5-HMF).

16. The method of claim 2, wherein the α-KG and the 5-HMF are present in the composition in a mass ratio of between 1:1 and 100:1.

17. The method of claim 2, wherein the composition comprises between 5 mg and 500 mg of the α-KG per kg of the subject's body weight and between 5 mg and 160 mg of the 5-HMF per kg of the subject's body weight.

18. The method of claim 2, comprising administering the composition:
(a) daily, as a single dose or divided into two or more equal or different doses;
(b) for at least 5 consecutive days; or
(c) 12 hours or less before exposure to ischemic or hypoxic conditions.

19. The method of claim 9, wherein the virus underlying the viral infection is a coronavirus.

20. The method of claim 11, wherein the contacting comprises completely immersing the organ or tissue into the preservation solution.

21. The method of claim 11, wherein the α-KG and the 5-HMF are in a concentration of:
(i) between 0.07 mg and 7 mg α-KG and between 0.07 mg and 2.24 mg 5-HMF per 1 ml of the or the preservation solution; or
(ii) between 0.005 mg and 0.5 mg α-KG and between 0.005 and 0.160 mg 5-HMF per 1 g of the organ or tissue.
